(19) United States
(12) Patent Application Publication
(10) Pub. No.: US 2025/0057566 A1
(43) Pub. Date: Feb. 20, 2025

(54) STABILIZATION DEVICE FOR ACCURATE NEEDLE INSERTION INTO CHEST PORTS

(71) Applicant: Hilario Castillo, Mount Vernon, NY (US)

(72) Inventor: Hilario Castillo, Mount Vernon, NY (US)

(21) Appl. No.: 18/772,148

(22) Filed: Jul. 13, 2024

Related U.S. Application Data
(60) Provisional application No. 63/533,356, filed on Aug. 18, 2023.

(51) Int. Cl. A61B 17/34; A61M 39/02

(52) U.S. Cl. CPC A61B 17/3403; A61M 39/02; A61M 2039/0205; A61M 2039/0258; A61M 2039/0288

(57) ABSTRACT

Disclosed is a stabilization device for accurate needle insertion into a chest port via septum. The stabilization device includes a left base portion, an elevated middle portion, a right base portion, and an opening. The elevated middle portion includes a left sidewall configured to extend from left base portion and to surround left sidewall of the chest port, a top cover is configured to extend from the left sidewall to cover the septum and a right sidewall is configured to extend from the top cover and configured to surround right sidewall of the chest port. The right base portion is configured to extend from the right sidewall and configured to position on right side of the chest port. The opening is configured on the elevated middle portion to align with the septum and configured to allow a user to accurately insert the needle into the chest port via the septum.

### CROSS REFERENCE TO RELATED APPLICATION

This application claims the benefit of U.S. Provisional Application No. 63/533,356 filed Aug. 18, 2023, the entirety of which is hereby incorporated by reference.

### BACKGROUND OF THE INVENTION

### 1. Field of the Invention

The present invention generally relates to medical devices and more specifically relates to a stabilization device for accurate needle insertion into chest ports.

### 2. Description of Related Art

Port-a-Caths, also known simply as ports, play a crucial role in modern medical treatments, particularly in patients who require frequent or long-term intravenous therapies. These devices are designed to provide a secure and reliable access point to a patient's bloodstream, enabling the administration of medications, fluids, and other treatments. Ports are especially valuable in cases where conventional intravenous access via peripheral veins is not feasible or practical.

Despite the benefits of Port-a-Caths, the conventional methods of accessing these devices using needles are not without challenges. During needle insertion, ports may move due to patient movement, anatomical variations, or other factors. This may result in multiple attempts and increased patient discomfort. Further, the movement of the port may lead to inaccurate needle insertion, requiring repeated attempts to access the port successfully. Furthermore, multiple attempts increase the time required for treatment administration, leading to inefficiencies in healthcare delivery. Moreover, repeated needle insertions increase the risk of complications, including infection, bleeding, and bruising, which may be detrimental to the patient's well-being.

Some existing methods attempt to address the challenges associated with port movement during needle insertion. These methods often involve manual techniques such as using the non-dominant hand to stabilize the port. Circular or triangular shapes are sometimes formed with fingers to provide support. However, these methods have limitations such as manual stabilization methods are imprecise and still result in port movement during needle insertion. Further, manual techniques are cumbersome for healthcare providers, as they require additional coordination and concentration during the procedure. The variability in manual stabilization techniques leads to inconsistent results in different healthcare settings.

The limitations of existing methods highlight the need for an innovative approach to address these challenges effectively and ensure accurate, comfortable, and efficient needle insertion into Port-a-Caths. Therefore, there is a need of a stabilization device for accurate needle insertion into chest ports. Further, the stabilization device should provide immobilization of the chest port during needle insertion.

### SUMMARY OF THE INVENTION

In accordance with teachings of the present invention, a stabilization device for accurate needle insertion into a chest port via septum, is provided.

An object of the present invention is to provide a stabilization device for accurate needle insertion into a chest port via septum. The stabilization device includes a left base portion, an elevated middle portion extending from the left base portion, a right base portion extending from the elevated middle portion, and an opening configured on the elevated middle portion.

Another object of the present invention is to allow a user to apply pressure from the left base portion and the right base portion to securely and accurately hold the chest port in its position during needle insertion.

Another object of the present invention is to provide a left indent configured on intersection of the left base portion and the elevated middle portion to provide a grip to hold the chest port firmly, and a right indent configured on intersection of the right base portion and the elevated middle portion to provide a grip to hold the chest port firmly.

Another object of the present invention is to provide a removable adhesive layer attached to bottom of the left base portion and the right base portion.

Another object of the present invention is a first step groove sidewall configured on bottom of the top cover to surround around top area of the chest port, and a second step groove sidewall configured to extend from the first step groove sidewall to surround the chest port. Further, the first step groove sidewall and the second step groove sidewall configured to securely and accurately hold the chest port in its position during needle insertion.

While a number of features are described herein with respect to embodiments of the inventions; features described with respect to a given embodiment also may be employed in connection with other embodiments. The following description and the annexed drawings set forth certain illustrative embodiments of the inventions. These embodiments are indicative, however, of but a few of the various ways in which the principles of the inventions may be employed. Other objects, advantages, and novel features according to aspects of the inventions will become apparent from the following detailed description when considered in conjunction with the drawings.

### BRIEF DESCRIPTION OF THE DRAWINGS

The annexed drawings, which are not necessarily to scale, show various aspects of the inventions in which similar reference numerals are used to indicate the same or similar parts in the various views;

FIG. 1 illustrates an exploded view of an arrangement to showcase placement of a stabilization device surrounding a chest port;

FIG. 2A illustrates a side perspective view of the stabilization device surrounding the chest port;

FIG. 2B illustrates a right side perspective view of the stabilization device placed on the chest port;

FIG. 3 illustrates a perspective view of stabilization device showing a user inserting needle into the chest port; and

FIG. 4 illustrates a rear perspective view of the stabilization device.

### DETAILED DESCRIPTION OF DRAWINGS

The present disclosure is now described in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

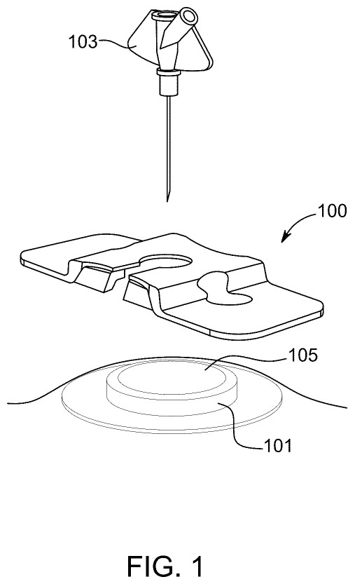

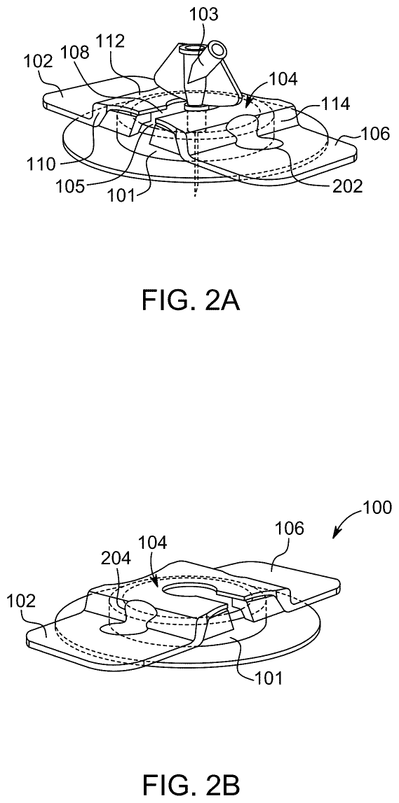

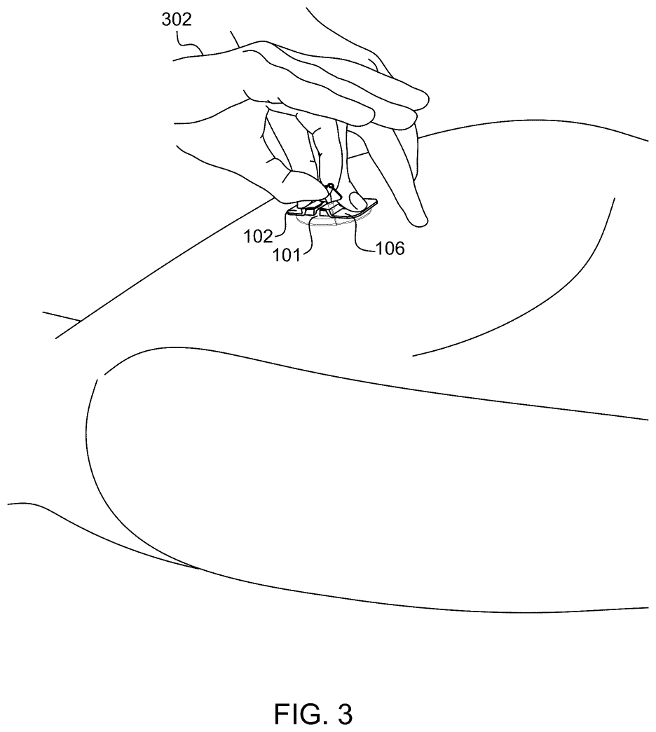

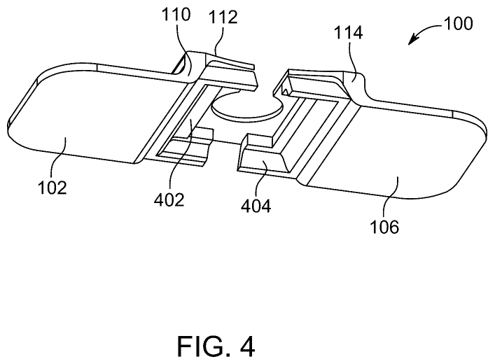

FIG. 1 illustrates an exploded view of an arrangement to showcase placement of a stabilization device 100 surrounding a chest port 101, in accordance with an embodiment of the present invention. The stabilization device 100 facilitates accurate needle 103 insertion into the chest port 101 via septum 105. The stabilization device 100 is explained in detail in conjunction with FIG. 2A, FIG. 2B, FIG. 3 and FIG. 4 of the present invention.

FIG. 2A illustrates a side perspective view of the stabilization device 100 placed on the chest port 101. The stabilization device 100 includes a left base portion 102, an elevated middle portion 104, a right base portion 106, and an opening 108. The left base portion 102 is configured to position on left side of the chest port 101. The left base portion 102 is explained in detail in conjunction with FIG. 2B of the present invention.

The elevated middle portion 104 includes a left sidewall 110, a top cover 112, and a right sidewall 114. The left sidewall 110 is configured to extend from the left base portion 102. The top cover 112 is configured to extend from the left sidewall to cover the septum 105.

The right sidewall 114 is configured to extend from the top cover 112. Further, the right sidewall 114 is configured to surround right sidewall of the chest port 101. The right base portion 106 is configured to extend from the right sidewall 114. Further, the right base portion 106 is configured to position on right side of the chest port 101.

In an embodiment, the left base portion 102, the elevated middle portion 104, and the right base portion 106 is configured in rectangular shape. It would be readily apparent to those skilled in the art that various shapes and sizes of the left base portion 102, the elevated middle portion 104 and the right base portion 106 may be envisioned such as square, planar, circular, without deviating from the scope of the present invention.

The opening 108 is configured on the elevated middle portion 104 to align with the septum 105. Further, the opening 108 configured to allow a user (shown in FIG. 3) to accurately insert the needle 103 into the chest port 101 via the septum 105. In a preferred embodiment, the opening 108 is configured to shape like a key-hole to allow easy access to the needle 103 into the chest port 101 via the septum 105.

The stabilization device 100 further includes a right indent 202 configured on intersection of the right base portion 106 and the elevated middle portion 104 to provide a grip to firmly hold the chest port 101. It would be readily apparent to those skilled in the art that various shapes and sizes of the right indent 202 such as finger shaped with printed impressions, may be envisioned without deviating from the scope of the present invention.

FIG. 2B illustrates a right side perspective view of the stabilization device 100 placed on the chest port 101. The left base portion 102 is configured to position on left side of the chest port 101. The stabilization device 100 further includes a left indent 204 configured on intersection of the left base portion 102 and the elevated middle portion 104 to provide a grip to firmly hold the chest port 101. It would be readily apparent to those skilled in the art that various shapes and sizes of the left indent 204 such as finger shaped with printed impressions, may be envisioned without deviating from the scope of the present invention.

FIG. 3 illustrates a perspective view of stabilization device 100 showing a user 302 inserting needle 103 into the chest port 101. The user 302 is able to securely and accurately hold the chest port 101 in its position by applying pressure through the left base portion 102 and the right base portion 106.

The user 302 applies pressure on the sidewalls of the elevated middle portion 104 through the left base portion 102 and the right base portion 106. The stabilization device 100 is crafted to accommodate the contours of the chest area of a patient, ensuring a secure fit over the chest port 101.

It would be readily apparent to those skilled in the art that various type of users 302 such as a nurse, doctor, surgeon, and various types of needle 101 such as hubber needle, non-coring needle may be envisioned without deviating from the scope of the present invention.

FIG. 4 illustrates a rear perspective view of the stabilization device 100 in accordance with an embodiment of the present invention. The stabilization device 100 includes a first step groove sidewall 402 is configured on bottom (inner surface) of the top cover 112 to surround top area of the chest port (101, shown in FIGS. 1 to 3).

Further, the stabilization device 100 includes a second step groove sidewall 404 is configured to extend from the first step groove sidewall 402 to surround sidewalls of the chest port (101, shown in FIGS. 1 to 3). The first step groove sidewall 402 and the second step groove sidewall 404 is configured to securely and accurately hold the chest port (101, shown in FIGS. 1 to 3) in its position during needle insertion.

The first groove sidewall 402 and the second groove sidewall 404 are configured to positon adjacent and between the left sidewall 110 and the right sidewall 114. The first groove sidewall 402 and the second groove sidewall 404 are configured to have C shape and reverse C shape extending from bottom of the top cover 112, adjacent to left sidewall 110, and the right sidewall 114.

Further in an embodiment, though not shown in Figures, the stabilization device 100 further includes a removable adhesive layer attached to bottom of the left base portion 102 and the right base portion 106. The user may remove the removable adhesive layer to stick the left base portion 102 and the right base portion 106 is attached to chest of the patient.

In an embodiment though not shown in Figures, the elevated middle portion having a top surface and a rear surface. The rear surface has contour engraving. Outer boundary of the septum fits into the contour engraving and centre portion of the septum is exposed through the opening for receiving the needle. Alternatively, the rear surface has the first groove sidewall and the second groove sidewall as shown in the FIG. 4. It would be readily apparent to those skilled in the art that various ways of securing the chest port to the elevated middle portion may be envisioned without deviating from the scope of the present invention.

The present invention offers various advantages such as allow a user (nurse or doctor) to firmly place the needle in the chest port. The stabilization device further ensures the chest port is held properly, stable and immobilize during needle insertion. The stabilization device assists in easy transition of fluids through needle into the chest port in processes like chemotherapy.

It would be readily apparent to those skilled in the art that various type of material such as medical-grade material may be envisioned to create the stabilization device 100 without deviating from the scope of the present invention. Further, the material should be sterilized capable, flexible plastics similar to silicone. The stabilization device is used for the chest port and similar devices like implantable ports, sub-cutaneous ports, or chemo-ports.

Although the inventions have been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the inventions. In addition, while a particular feature of the inventions may have been described above with respect to only one or more of several illustrated embodiments, such feature may be combined with one or more other features of the other embodiments, as may be desired and advantageous for any given or particular application.

While the invention has been described with respect to a limited number of embodiments, those skilled in the art, having benefit of the above description, will appreciate that other embodiments may be devised which do not 10 depart from the scope of the present invention as described herein. Accordingly, the scope of the present invention should be limited only by the appended claims.

1. A stabilization device for accurate needle insertion into a chest port via septum, the stabilization device comprising:
a left base portion configured to position on left side of the chest port;
an elevated middle portion comprising:
a left sidewall configured to extend from the left base portion, further the left sidewall configured to surround left sidewall of the chest port;
a top cover configured to extend from the left sidewall to cover the septum;
a right sidewall configured to extend from the top cover, further the right sidewall configured to surround right sidewall of the chest port;
a right base portion configured to extend from the right sidewall, further the right base portion configured to position on right side of the chest port; and
an opening configured on the elevated middle portion to align with the septum, further the opening configured to allow a user to accurately insert the needle into the chest port via the septum.

2. The stabilization device of claim 1, wherein the user applies pressure from the left base portion and the right base portion to securely and accurately hold the chest port in its position during needle insertion.

3. The stabilization device of claim 1 further comprising a left indent configured on intersection of the left base portion and the elevated middle portion to provide a grip to hold the chest port firmly.

4. The stabilization device of claim 1 further comprising a right indent configured on intersection of the right base portion and the elevated middle portion to provide a grip to firmly hold the chest port.

5. The stabilization device of claim 1 further comprising a removable adhesive layer attached to bottom of the left base portion and the right base portion.

6. The stabilization device of claim 1 wherein the opening is configured to receive a Huber needle.

7. The stabilization device of claim 1 further comprising a first step groove sidewall configured on bottom of the top cover to surround top area of the chest port.

8. 8. The stabilization device of claim 7 further comprising a second step groove sidewall configured to extend from the first step groove sidewall to surround the chest port.

9. The stabilization device of claim 8 wherein the first step groove sidewall and the second step groove sidewall configured to securely and accurately hold the chest port in its position during needle insertion.
